(19) United States
(12) Patent Application Publication
(10) Pub. No.: US 2006/0089302 A1
(43) Pub. Date: Apr. 27, 2006

(54) Hsp70-derived peptides and uses thereof in the diagnosis and treatment of autoimmune diseases

(76) Inventors: Rivkn Abulafia-Lapid, Yahud (IL); Henri Atlan, Jerusalem (IL); Irun R Cohen, Rehovot (IL)

Correspondence Address: MERCHANT & GOULD PC, P.O. BOX 2903, MINNEAPOLIS, MN, 55402-0903, US

(21) Appl. No.: 10/505,848

(22) PCT Filed: Feb. 24, 2003
(86) PCT No.: PCT/IL03/00143

§ 371 Date: May 10, 2005

(30) Foreign Application Priority Data
Feb. 26, 2002 (IL) 148401

(51) Int. Cl. A61K 39/00; C07K 14/47; C12Q 1/68; G01N 33/53

(52) U.S. Cl. 514/12; 530/350; 435/7.1; 435/6

(57) ABSTRACT

The invention relates to specific peptides derived from hsp70, and to pharmaceutical compositions comprising the same. The peptides and compositions of the invention are particularly suitable for the prevention or treatment of an autoimmune disease such as Type 1 Diabetes, Systemic Lupus Erithematosus, Multiple Sclerosis or Rheumatoid Arthritis. The invention further relates to a method for diagnosing the occurrence or incipience of an autoimmune disease in a patient by use of the peptides of the invention, by testing a blood or urine sample of a patient for the presence of antibodies or T-cells which are immunologically reactive to human hsp70. The invention also relates to a kit for the diagnosis of an autoimmune disease by testing for the presence of anti-hsp70 antibodies by aid of the peptides of the invention.

### FIELD OF THE INVENTION

The present invention relates to methods of treatment and diagnosis of autoimmune diseases. More specifically, the invention relates to hsp70 peptides and their use in the diagnosis and treatment of autoimmune diseases.

### BACKGROUND OF THE INVENTION

Type 1 Diabetes (Insulin dependent diabetes mellitus, IDDM) is a disease caused by autoimmune T-cells that attack the insulin-producing β cells of the pancreatic islets [Bach, J. F. (1994) Endocrine Reviews 15:516-542; Atkinson, M. A. and Maclaren, N. K. (1994) New Engl. J. Med. 331:1428-1436; Honeyman, M. C. and Harrison, L. C. (1993) Springer Semin. Immunol pathol. 14(3):253-274]. In humans and in the NOD mice model system, in which the condition develops spontaneously, the disease appears to involve autoimmunity to a similar collective of antigens including proinsulin and insulin [Honeyman, M. C., and Harrison, L. C. (1993) id ibid.; Roep B. O. (1996) Diabetes 45:1147-1156], glutamic acid decarboxylase (GAD) [Harrison, L. C. et al. (1991) Diabetes 40 (9):1-128-1133; Naquet, P. et al. (1988) J. Immunol. 140:2569-2578], the isle T-cell antigen ICA69 [Atkinson, M. A. et al. (1992) Lancet 339:458-459], and the insulin secretory-granule 38 kDa protein (38 kDa) [Atkinson, M. A. et al. (1994) J. Clin. Invest. 94:2125-2129; reviewed in: Atkinson, M. A. and Maclaren, N. K. (1994) id ibid.]. In addition, the 60 kDa heat-shock protein (hsp60) is one of the auto-antigens found in the NOD mouse model.

It is interesting that Type 1 diabetes patients and NOD mice appear to make T-cell responses to similar hsp60 peptides. The similarity in target peptides may result from the similar peptide-binding motifs of the mouse I-Ag7 [Reizis, B. et al. (1996) Internation. Immunol. 9 (1): 43-51] and the human DQ8 MHC molecules [Kwok, W. W. et.al. (1996) J. Immunol. 156:2171-7], both associated with susceptibility to IDDM.

Studies in the NOD mouse model indicate that Type 1 Diabetes is a T-cell mediated disease, wherein the cells involved in the pathogenesis of the disease are Th1-type T-cells. It has been shown that NOD mice spontaneously develop T-cells responsive to a hsp60 peptide, and these T-cells can adoptively transfer diabetes or, when attenuated, can vaccinate mice against diabetes (T-cell vaccination, TCV) [Elias, D. et al. (1991) Proc. Natl. Acad. Sci. USA 88:3088-91]. Moreover, a single, subcutaneous administration of a hsp60 peptide either early, at 4-6 weeks of age [Elias, D. et al. (1991) id ibid.], or very late in the autoimmune process, at 12-17 weeks, can arrest the disease [Elias, D. et al. (1994) Lancet 343:704-706; Elias, D. and Cohen, I. R. (1995) Diabetes 44:1132-1138]. The same hsp60 peptide was also found to influence toxin-induced diabetes. Mice of the C57BL/KsJ strain can be induced to develop a type of autoimmune diabetes about 3 months after administration of a very low dose of the β-cell toxin streptozotocin [Elias, D. et al. (1994) Diabetes 43:992-998]. This form of diabetes could also be treated with the hsp60 peptide administered after the toxic insulitis. In contrast to the hsp60 peptide treatment, treatment of the mice with an immunogenic GAD peptide failed to arrest the development of diabetes [Elias, D. and Cohen, IR. (1996) Diabetes 45:1168-1172]. Effective treatment of the diabetic process in mice with hsp60 peptides appears to involve a temporary burst of “anti-inflammatory” Th2-like reactivity that down-regulates pathogenic Th1-like reactivity to hsp60. This down-regulation induced by hsp60 appears to spread to down-regulate the Th1-like responses to other antigens targeted in Type 1 Diabetes [Elias, D. et al. (1997) Diabetes 46:758-764].

Anti-hsp60 T-cells can also mediate insulitis and hyperglycemia [Roep, B. O. et al. (1996) Euro. J. Immunol. 26(6):1285-1289], and modulating the anti-hsp60 T-cell response can lead to the arrest of the autoimmune destruction of β cells [Roep B. O. et al. (1991) Lancet 337:1439-1441; Elias, D. et al. (1991) id ibid.]. Recently, the inventors reported proliferative responses to human hsp60 and its peptides in 25 newly diagnosed Type 1 Diabetes adult patients amongst whom 92% tested positive to hsp60 [Abulafia-Lapid, R. et al. (1999) J. Autoimmunity 12:121-129].

Several studies have suggested that other heat shock proteins, like hsp70 and hsp90, may also have a role in the pathogenesis of autoimmune disorders [Lindquist, S. (1988) Annu. Rev. Genet. 22:631-677; Polla, B. S. and Young, D. (1989) Immunol. Today 10:393-394; Feige, U. and van Eden, W. (1996) Infection, autoimmunity and autoimmune disease EXS 77:359-373]. A role for these molecules in antigen presentation has been reported [Kaufman, S. H. E. (1990) Immunol. Today 11:129-136; Van Buskirk, A. et al. (1989) J. Exp. Med. 170:1799-1809], as well as the association of the 8.5 kD hsp70-2 allele with diabetic haplotypes [Pugliese, A. et al. (1992) Diabetes 41:788-791]. Moreover, auto-antibodies against hsp70 and hsp90 have been found in patients suffering from systemic lupus erythematosus, polymyositis [Minota, S. and Winfield, J. (1988) Arthritis Rheum. 31:S13; Minota, S. et al. (1988) J. Clin. Invest. 81:106-119] and multiple sclerosis [Salvetti M. et al. (1996) J. Neuroimmunol. 65(2):143-153]. Therefore, it has been further suggested that hsp70 and hsp90 might also be target antigens in Type 1 Diabetes [Minota, S. et al. (1988) id ibid.].

In view of these suggested roles for hsp70, the inventors investigated whether children newly diagnosed with Type 1 Diabetes present T-cell proliferative responses to human hsp70 and hsp90. The inventors found that in children who were newly diagnosed as having Type 1 diabetes, there was a T-cell proliferative response to hsp70 but not to hsp90. It is important that this response was measured in newly diagnosed children, since the T-cell response is acute and destructive until the α-cell islets are destroyed, as demonstrated by the T-cell response to hsp60 protein, which declines at about 16 weeks after diagnosis [Abulafia-Lapid et al. (1999) id ibid.]. In addition, the inventors tested the specific response to hsp70 peptides, while mapping the major hsp70 protein epitopes. Finally, the presence of sera antibodies to hsp60, hsp70, and hsp90 was tested.

In view of the interesting results obtained with respect to the anti-hsp70 response, the inventors have developed methods for the diagnosis and treatment of autoimmune diseases in general, and more specifically to Type 1 Diabetes, utilizing for this purpose the hsp70 peptides of the invention.

It is therefore an object of the present invention to provide novel hsp70 peptides. It is also an object of the invention to provide methods of diagnosis and treatment of autoimmune diseases using the novel hsp70 peptides of the invention.

These and other objects of the invention will become more apparent as the description proceeds.

### SUMMARY OF THE INVENTION

The present invention relates to hsp70 peptides and their use in the diagnosis and treatment of autoimmune diseases, preferably Type 1 Diabetes, Systemic Lupus Erithematosus, Multiple Sclerosis or Rheumatoid Arthritis, more preferably Type 1 Diabetes.

As presently claimed, in a first aspect, the present invention relates to a peptide selected from the group consisting of the peptides denoted by SEQ. ID. NO.1, SEQ. ID. NO.2, SEQ. ID. NO.3, SEQ. ID. NO.4, SEQ. ID. NO.5, SEQ. ID. NO.6, SEQ. ID. NO.7, SEQ. ID. NO.8, SEQ. ID. NO.9, SEQ. ID. NO.10, SEQ. ID. NO.11, SEQ. ID. NO.12, SEQ. ID. NO. 13, SEQ. ID. NO.14, SEQ. ID. NO.15, SEQ. ID. NO.16, SEQ. ID. NO.17, SEQ. ID. NO.18, SEQ. ID. NO.19, SEQ. ID. NO.20, SEQ. ID. NO.21, SEQ. ID. NO.22, SEQ. ID. NO.23, SEQ. ID. NO.24, SEQ. ID. NO.25, SEQ. ID. NO.26, SEQ. ID. NO.27, SEQ. ID. NO.28, SEQ. ID. NO.29, SEQ. ID. NO.30, SEQ. ID. NO.31, SEQ. ID. NO.32, SEQ. ID. NO.33, SEQ. ID. NO.34, SEQ. ID. NO.35, SEQ. ID. NO.36, SEQ. ID. NO.37, SEQ. ID. NO.38, SEQ. ID. NO.39, SEQ. ID. NO.40, SEQ. ID. NO.41, SEQ. ID. NO.42 and SEQ. ID. NO.43, and salts, analogues and functional derivatives thereof. Preferably, the peptide of the invention is selected from the group consisting of the peptides SEQ. ID. NO.1, SEQ. ID. NO.12, SEQ. ID. NO.15, SEQ. ID. NO.16, SEQ. ID. NO.19, SEQ. ID. NO.27, SEQ. ID. NO.29, SEQ. ID. NO.34 and SEQ. ID. NO.35. More preferably, the peptide of the invention is selected from the group consisting of the peptides denoted by SEQ. ID. NO.1, SEQ. ID. NO.27 and SEQ. ID. NO.35.

Functional derivatives of the peptide of the invention consist of chemical modifications to amino acid side chains and/or the carboxyl and/or amino moieties of said peptides.

In a second aspect, the present invention relates to a pharmaceutical composition comprising at least one peptide of the invention, and optionally comprising a pharmaceutically acceptable carrier.

In a specific embodiment, the pharmaceutical composition of the invention is for use in the prevention or treatment of an autoimmune disease, preferably Type 1 Diabetes, Systemic Lupus Erithematosus, Multiple Sclerosis or Rheumatoid Arthritis, more preferably Type 1 Diabetes.

In a third aspect, the present invention relates to a method for diagnosing the occurrence or incipience of an immune disease in a patient, utilizing a peptide as defined in the first aspect of the invention. Preferably, the immune disease is Type 1 Diabetes, Systemic Lupus Erithematosus, Multiple Sclerosis or Rheumatoid Arthritis. More preferably, the immune disease is Type 1 Diabetes.

In one embodiment, the method of the invention comprises testing a blood or urine sample of said patient for the presence of antibodies or T-cells which are immunologically reactive to human hsp70. Said method involves contacting said sample with a peptide of the invention and detecting an immunoreaction between said sample and said peptide, wherein the presence of such immunoreaction indicates the presence of anti-hsp70 antibodies or of a T-cell which immunoreacts with hsp70, indicating an increased probability of the presence or incipience of an autoimmune disease.

In a specific embodiment, the presence of anti-hsp70 antibodies is revealed by an immunoreaction detected by radioimmunoassay and/or by an ELISA test or any other test that might detect the said anti-hsp70.

In a further specific embodiment, the method to test for the presence of said T-cell which immunoreacts with hsp70 comprises the steps of:
(a) preparing a mononuclear cell fraction containing T-cells from a blood sample obtained from said patient;
(b) adding to said mononuclear cell fraction an antigen selected from the peptides defined in the invention;
(c) incubating said cell fraction in the presence of said antigen for a suitable period of time and under suitable culture conditions;
(d) adding a labeled nucleotide to the incubated cell culture of (c) at a suitable time before the end of said incubation period to provide for the incorporation of said labeled nucleotide into the DNA of proliferating T-cells; and
(e) determining by suitable means the amount of proliferating T-cells by analysis of the amount of labeled nucleotide incorporated into said T-cells.

In a further aspect, the invention relates to a kit for the diagnosis of an autoimmune disease. Preferably the kit is for the diagnosis of Type 1 Diabetes, Systemic Lupus Erithematosus, Multiple Sclerosis or Rheumatoid Arthritis. More preferably, said kit is for the diagnosis of Type 1 Diabetes.

In one embodiment, said diagnosis is achieved by testing for the presence of anti-hsp70 antibodies, wherein said kit comprises the following components:
(a) at least one antigen selected from peptides of the invention; and
(b) a tagged antibody capable of recognizing the non-variable region of said anti-hsp70 antibodies.

In another embodiment, said diagnosis is achieved by testing for the presence of a T-cell which immunoreacts with hsp70, wherein said kit comprises the following components:
(a) at least one antigen selected from the peptides of the invention;
(b) a suitable medium for culture of lymphocytes (T-cells); and
(c) a labeled nucleotide for a T-cell proliferation test.

In a last aspect, the invention relates to a method of modulating an immune response, and arresting the autoimmune process, in a patient in need of such treatment, wherein said method comprises administering to said patient, at least once, a peptide selected from the peptides of the invention in a medically effective amount. Said method shall help the body to stop the auto-immune process.

### BRIEF DESCRIPTION OF THE FIGURES

The present invention will be more clearly understood from the detailed description of the preferred embodiments and from the attached figures in which:

FIG. 1: T-cell responses of 25 Type 1 Diabetes children to the intact hsp60 and hsp70 molecules

T-cells were activated with 2-5 μg/ml hsp60 and 2-5 μg/ml hsp70. The proliferative responses are presented in arbitrary S.I. (stimulation index) units. A T-cell proliferation of S.I.≧2 was considered positive, and this cut off is marked by a horizontal dashed line. Abbreviations: pat., patients.

FIG. 2A-B: Epitope mapping of hsp70 protein

Seven Type 1 Diabetes patients (P3, P16, P18, P20, P21, P23 and P24) were tested for responsiveness to multiple hsp70 epitopes that encompassed the entire human hsp70 molecule. T-cells isolated from the seven representative patients were assayed for proliferative responses to the 43 overlapping hsp70 peptides (detailed in Table 2). T-cells were activated with 5-20 μg/ml of each peptide. A T-cell proliferation of S.I.≧2 was considered positive.

FIG. 2A: Responses to peptides p1-p22.

FIG. 2B: Responses to peptides p23-p43.

Abbreviations: pat., patients; pept., peptides.

FIG. 3: Measurement of sera auto-antibodies to hsp60, hsp70 and hsp90.

This graph represents the levels of IgG antibodies to hsp60, hsp70 and hsp90 in the sera of 21 patients with Type 1 Diabetes (Table 1A) in comparison with the sera of 10 normoglycemic children. The level of antibodies was considered positive when it was greater than the mean of the values of antibody levels obtained from the control group (normoglycemic children) plus two standard deviations. The mean of the values from the control group plus two standard deviations was thus considered as the cut-off level. Abbreviations: pos., positives; antig., antigen; Diab. I, Type 1 Diabetes; healt., healthy; Tet. Tox., Tetanus Toxoid.

### DETAILED DESCRIPTION OF THE INVENTION

For purposes of clarification, the following terms are defined herein:
hsp: heat-shock protein.
IDDM: Insulin-dependent Diabetes Mellitus, recently denominated Type 1 Diabetes.
NIDDM: Non-insulin Dependent Diabetes Mellitus, recently denominated Type 2 Diabetes.
NOD mice: Non-Obese Diabetes Mice. A mouse model that develops a spontaneous form of diabetes, considered a good model for Type 1 Diabetes. Female NOD mice develop insulitis at around 4 weeks of age and hyperglycemia starts at about 14-17 weeks. By 35-40 weeks almost all female NOD mice have developed severe diabetes and most die in the absence of insulin treatment.
PBMC: peripheral blood mononuclear cells.
S.I.: stimulation index, this index is calculated by dividing the response (in cpm counts) by the cpm counts obtained in the background (which is set by the counts given by T cells in culture in the absence of the antigen).
TT: tetanus toxoid.

The inventors have found that a significant proportion of recently diagnosed Type 1 Diabetes children manifest T-cell proliferative activity to human hsp60 and hsp70 proteins, but not to hsp90. Most importantly, comparing the T-cell response to each one of hsp60, hsp70 and hsp90, (referred to in the Examples as the Stimulation Index, S.I.), the inventors observed that the response to hsp70 was the highest in Type 1 Diabetes patients (Example 1, Table 1A).

Curiously, subjects with Type 2 Diabetes did not show higher response to either hsp60 or hsp70 (Example 1, Table 1C), than did healthy subjects (Example 1, Table 1B). Therefore, hsp70 may be a member of the collective of self-antigens to which there is enhanced T-cell reactivity in Type 1 Diabetes, but not in Type 2 Diabetes [Roep, B. (1996) id ibid.].

The present invention thus relates to methods of treatment and diagnosis of an autoimmune disease. More specifically, the invention relates to hsp70 peptides and their use in the diagnosis and treatment of said autoimmune disease. Preferably, the autoimmune disease to be diagnosed or treated is Type 1 Diabetes, Systemic Lupus Erithematosus, Multiple Sclerosis or Rheumatoid Arthritis. More preferably, said autoimmune disease is Type 1 Diabetes.

In a first aspect, the present invention relates to hsp70 overlapping peptides, selected from the group consisting of peptides denoted by any one of SEQ.ID.NO.1, SEQ.ID.NO.2, SEQ.ID.NO.3, SEQ.ID.NO.4, SEQ.ID.NO.5, SEQ.ID.NO.6, SEQ.ID.NO.7, SEQ.ID.NO.8, SEQ.ID.NO.9, SEQ.ID.NO.10, SEQ.ID.NO.11, SEQ.ID.NO.12, SEQ.ID.NO.13, SEQ.ID.NO.14, SEQ.ID.NO.15, SEQ.ID.NO.16, SEQ.ID.NO.17, SEQ.ID.NO.18, SEQ.ID.NO.19, SEQ.ID.NO.20, SEQ.ID.NO.21, SEQ.ID.NO.22, SEQ.ID.NO.23, SEQ.ID.NO.24, SEQ.ID.NO.25, SEQ.ID.NO.26, SEQ.ID.NO.27, SEQ.ID.NO.28, SEQ.ID.NO.29, SEQ.ID.NO.30, SEQ.ID.NO.31, SEQ.ID.NO.32, SEQ.ID.NO.33, SEQ.ID.NO.34, SEQ.ID.NO.35, SEQ.ID.NO.36, SEQ.ID.NO.37, SEQ.ID.NO.38, SEQ.ID.NO.39, SEQ.ID.NO.40, SEQ.ID.NO.41, SEQ.ID.NO.42, SEQ.ID.NO.43. Preferably, the peptides of the invention are selected from the group consisting of peptides denoted by any one of SEQ.ID.NO.1, SEQ.ID.NO.12, SEQ.ID.NO.15, SEQ.ID.NO.16, SEQ.ID.NO.19, SEQ.ID.NO.27, SEQ.ID.NO.29, SEQ.ID.NO.34 and SEQ.ID.NO.35. More preferably, the peptides of the invention are the peptides denoted by SEQ.ID.NO.1, SEQ.ID.NO.27 and SEQ.ID.NO.35.

The peptides of the invention may be used in free form or as salt, e.g., as metal salt, including sodium, potassium, lithium or calcium salt, or as a salt with an organic base, or as a salt with a mineral acid, including sulfuric acid, hydrochloric acid or phosphoric acid, or with an organic acid e.g., acetic acid or maleic acid. Generally, any pharmaceutically acceptable salt of the peptide of the invention may be used, as long as the biological activity of the peptide with respect to diabetes is maintained.

Functional derivatives consist of chemical modifications to amino acid side chains and/or the carboxyl and/or amino moieties of said peptides. Modifications can also include backbone modifications, like insertions, deletions or replacement of any one of the amino acids of said peptides.

It is to be understood by all of skill in the art that suitable analogs of these new peptides may be readily synthesized by now-standard peptide synthesis methods and apparatus. The only limitation on such analogs is that they have essentially the same biological activity of the hsp70 peptides with respect to diabetes. All such analogs will essentially be based on the new peptides as regards their amino acid sequence but will have one or more amino acid residues deleted, substituted or added. When amino acid residues are substituted, such substitutions which are envisaged are those which do not significantly alter the structure or biological activity of the peptide, for example basic amino acids will be replaced with other basic amino acids, acidic ones with acidic ones and neutral ones with neutral ones. The overall length of the analog peptide can be between about 9 to 35 amino acids. Preferably, when amino acid residues are deleted, the same above restraints are applied as regards obtaining the aforesaid biologically active peptides, and also wherein such deletion analogs will still have between about 17 to about 23 amino acid residues (deletion analogs will usually be no less than about 13 amino acid residues in length). Further preferably, when amino acid residues are added, the aforesaid restraints concerning biological activity are applied and such addition analogs will usually still have between about 17 to about 23 amino acids (addition analogs will usually be up to about 30 amino acids in length).

In a second aspect, the invention relates to a pharmaceutical composition comprising a peptide selected from the group consisting of peptides denoted by any one of SEQ.ID.NO.1, SEQ.ID.NO.12, SEQ.ID.NO.15, SEQ.ID.NO.16, SEQ.ID.NO.19, SEQ.ID.NO.27, SEQ.ID.NO.29, SEQ.ID.NO.34 and SEQ.ID.NO.35. Preferably, the pharmaceutical composition of the invention comprises a peptide selected from the group consisting of SEQ.ID.NO.1, SEQ.ID.NO.27 and SEQ.ID.NO.35. The pharmaceutical compositions of the invention may also comprise a mixture of at least two of the peptides denoted by any one of SEQ.ID.NO.1, SEQ.ID.NO.12, SEQ.ID.NO.15, SEQ.ID.NO.16, SEQ.ID.NO.19, SEQ.ID.NO.27, SEQ.ID.NO.29, SEQ.ID.NO.34 and SEQ.ID.NO.35. Optionally, the pharmaceutical composition also comprises pharmaceutically acceptable carrier, additive and/or diluent.

The peptides of the invention may be used as such or in the form of a composition. A composition will generally contain salts, preferably in physiological concentration, such as PBS (phosphate-buffered saline), or sodium chloride (0.9% w/v), and a buffering agent, such as phosphate buffer in the above PBS. The preparation of pharmaceutical compositions is well known in the art, see e.g., U.S. Pat. Nos. 5,736,519, 5,733,877, 5,554,378, 5,439,688, 5,418,219, 5,354,900, 5,298,246, 5,164,372, 4,900,549, 4,755,383, 4,639,435, 4,457,917, and 4,064,236. The peptide of the present invention, or a pharmacologically acceptable salt thereof is preferably mixed with an excipient, carrier, diluent, and optionally, a preservative or the like pharmacologically acceptable vehicles as known in the art, see e.g., the above US patents. Examples of excipients include glucose, mannitol, inositol, sucrose, lactose, fructose, starch, corn starch, microcrystalline cellulose, hydroxypropylcellulose, hydroxypropylmethyl-cellulose, polyvinyl-pyrrolidone and the like. Optionally, a thickener may be added, such as a natural gum, a cellulose derivative, an acrylic or vinyl polymer, or the like.

The pharmaceutical composition is provided in solid, liquid or semi-solid form. A solid preparation may be prepared by blending the above components to provide a powdery composition. Alternatively, the pharmaceutical composition is provided as lyophilized preparation. The liquid preparation is provided preferably as aqueous solution, aqueous suspension, oil suspension or microcapsule composition. A semi-solid composition is provided preferably as hydrous or oily gel or ointment.

A solid composition may be prepared by mixing an excipient with a solution of the peptide of the invention, gradually adding a small quantity of water, and kneading the mixture. After drying, preferably in vacuum, the mixture is pulverized. A liquid composition may be prepared by dissolving, suspending or emulsifying the peptide of the invention in water, a buffer solution or the like. An oil suspension may be prepared by suspending or emulsifying the peptide of the invention or protein in an oleaginous base, such as sesame oil, olive oil, corn oil, soybean oil, cottonseed oil, peanut oil, lanolin, petroleum jelly, paraffin, Isopar, silicone oil, fatty acids of 6 to 30 carbon atoms or the corresponding glycerol or alcohol esters. Buffers include Sorensen buffer (Ergeb. Physiol., 12, 393 1912), Clark-Lubs buffer (J. Bact., 2, (1), 109 and 191, 1917), Macllvaine buffer (J. Biol. Chem., 49, 183, 1921), Michaelis buffer (Die Wasserstoffinonenkonzentration, p. 186, 1914), and Kolthoff buffer (Biochem. Z., 179, 410, 1926).

A composition may be prepared as a hydrous gel, e.g. for transnasal administration. A hydrous gel base is dissolved or dispersed in aqueous solution containing a buffer, and the peptide of the invention, and the solution warmed or cooled to give a stable gel.

Preferably, the peptide of the invention is administered through intravenous, intramuscular or subcutaneous administration. Oral administration is expected to be less effective, because the peptide may be digested before being taken up. Of course, this consideration may apply less to a peptide of the invention which is modified, e.g., by being cyclic peptide, by containing non-naturally occurring amino acids, such as D-amino acids, or other modification which enhance the resistance of the peptide to biodegradation. Decomposition in the digestive tract may be lessened by use of certain compositions, for instance, by confining the peptide of the invention in microcapsules such as liposomes. The pharmaceutical composition of the invention may also be administered to other mucous membranes. The pharmaceutical composition is then provided in the form of a suppository, nasal spray or sublingual tablet. The dosage of the peptide of the invention may depend upon the condition to be treated, the patient's age, bodyweight, and the route of administration, and will be determined by the attending physician.

In another embodiment, the peptide of the invention may be provided in a pharmaceutical composition comprising a biodegradable polymer selected from poly-1,4-butylene succinate, poly-2,3-butylene succinate, poly-1,4-butylene fumarate and poly-2,3-butylene succinate, incorporating the peptide of the invention as the pamoate, tannate, stearate or palmitate thereof. Such compositions are described e.g., in U.S. Pat. No. 5,439,688.

In another embodiment, a composition of the invention is a fat emulsion. The fat emulsion may be prepared by adding to a fat or oil about 0.1-2.4 w/w of emulsifier such as a phospholipid, an emulsifying aid, a stabilizer, mixing mechanically, aided by heating and/or removing solvents, adding water and isotonic agent, and optionally, adjusting adding the pH agent, isotonic agent. The mixture is then homogenized. Preferably, such fat emulsions contain an electric charge adjusting agent, such as acidic phospholipids, fatty acids, bilic acids, and salts thereof. Acidic phospholipids include phosphatidylserine, phosphatidylglycerol, phosphatidylinositol, and phosphatidic acid. Bilic acids include deoxycholic acid, and taurocholic acid. The preparation of such pharmaceutical compositions is described in U.S. Pat. No. 5,733,877.

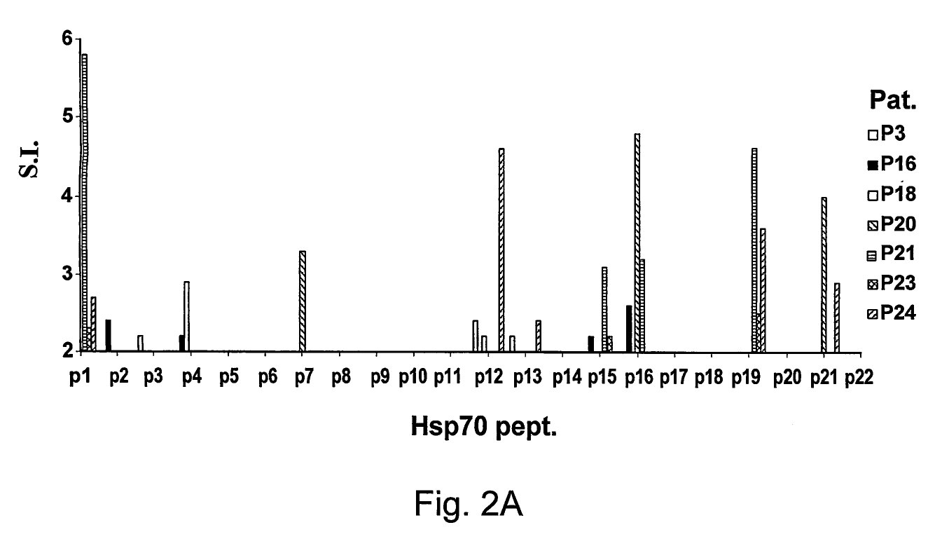

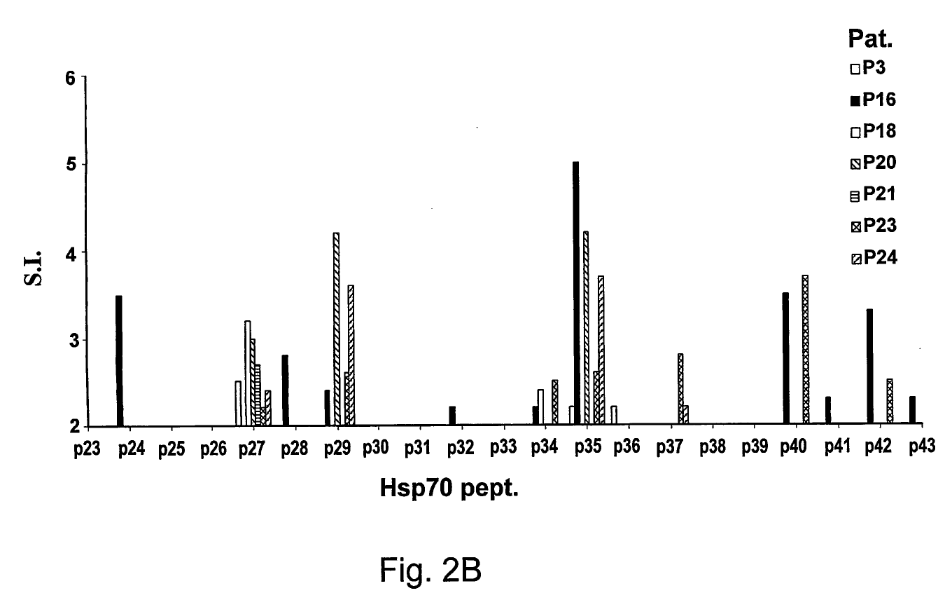

The inventors have mapped the responses of patients to a series of overlapping hsp70 peptides. FIG. 2 shows that hsp70 peptides p1, p12, p15, p16, p19, p27, p29, p34 and p35 triggered a T-cell response in the Type 1 Diabetes patients tested.

The peptides of the invention contain (with the exception of p43) 20 amino acids. It is known that the immunogenic motif that is recognized by the antigen-presenting cell (APC), which display the HLA class II, is only 9 amino acids long.

The auto-reactive T-cells involved in the autoimmune process are activated by APCs. The APC processes the self-antigen and presents the immuno-dominant peptide on the MHC (Major Histocompatibility Complex, in human known as HLA), which then becomes available for recognition by antigen-specific T-cells. Interestingly, certain HIA molecules have been shown to be associated with auto-immunity due to the presentation of disease-associated peptides. For example, the HLA-DQB1*0302 has been described as associated with Type 1 Diabetes, for preferentially binding to GAD (glutamic acid decarboxylase), a protein that has been associated with the disease [W. W. et al. (1996) id ibid.]. Therefore, it remains to be determined which HLA haplotypes are expressed by the Type 1 Diabetes patients studied in the present invention, to understand how these haplotypes correlate with the specific hsp70 peptides recognized by the patients.

In one embodiment, the pharmaceutical composition is intended for the prevention or treatment of an autoimmune disease, preferably Type 1 Diabetes, Systemic Lupus Erithematosus, Multiple Sclerosis or Rheumatoid Arthritis, and more preferably Type 1 Diabetes. Type 1 Diabetes has also been known as Insulin-Dependent Diabetes Mellitus (IDDM).

Another aspect of the present invention relates to a method for diagnosing an autoimmune disease in a patient, also at an incipient stage. Preferably, said autoimmune disease is Type 1 Diabetes, Systemic Lupus Erithematosus, Multiple Sclerosis or Rheumatoid Arthritis. More preferably, the autoimmune disease is Type 1 Diabetes. The diagnostic method of the invention comprises testing a biological sample obtained from a patient, preferably blood or urine sample of said patient, using a peptide of the invention as an antigen, to detect the presence of antibodies or T-cells which are immunologically reactive to human hsp70, whereby the presence of anti-hsp70 antibodies or of a T-cell which immunoreacts with hsp70 indicates an increased probability of the presence or incipience of an autoimmune disease.

In one embodiment, the method of testing for the presence of anti-hsp70 antibodies comprises a radioimmunoassay or an ELISA test.

In a second embodiment of said method of diagnosis, the patient is tested for the presence of a T-cell which immunoreacts with hsp70, comprising the steps of: (a) preparing a mononuclear cell fraction containing T-cells from a blood sample obtained from said patient; (b) adding to said mononuclear cell fraction an antigen selected from the peptides of the invention; (c) incubating said cell fraction in the presence of said antigen for a suitable period of time and under suitable culture conditions; (d) adding a labeled nucleotide to the incubated cell culture of (c) at a suitable time before the end of said incubation period to provide for the incorporation of said labeled nucleotide into the DNA of proliferating T-cells; and (e) determining the amount of proliferating T-cells by analysis of the amount of labeled nucleotide incorporated into said T-cells by suitable means.

The diagnostic differentiation of Type 1 diabetes from Type 2 diabetes is of major importance. Type 2 diabetes is not an autoimmune disease, and is usually treated by the oral administration of insulin. Misdiagnosis of Type 1 for Type 2 diabetes, since the treatment designed for slow-release of insulin could worsen the condition and lead to shock, and in some case, particularly in children, could be life-threatening. The present diagnostic method affords a reliable diagnosis of the Type 1 diabetes, and thus avoiding any risks which may result from mis-diagnosis.

The invention also provides a kit for the diagnosis of an autoimmune disease, by testing reactivity to hsp70 antibodies in patients and suspected patients. Preferably, the autoimmune disease to be diagnosed is Type 1 Diabetes, Systemic Lupus Erithematosus, Multiple Sclerosis or Rheumatoid Arthritis. More preferably, said autoimmune disease is Type 1 Diabetes.

In one embodiment, said kit provides means for conducting a test for the presence of anti-hsp70 antibodies, and comprises the following components: (i) at least one antigen selected from the peptides of the invention; and (ii) means for detecting said anti-hsp70 antibodies, for example a labeled antibody capable of recognizing the non-variable region of the anti-hsp70 antibodies.

In a second embodiment, the kit provides means for conducting a test for the presence of a T-cell which immunoreacts with hsp70. In this embodiment, the kit comprises the following components: (i) at least one antigen selected from the peptides of the invention; (ii) a suitable medium for culture of lymphocytes (T-cells); and (iii) means for detecting T-cell proliferation, e.g. a labeled nucleotide for a T-cell proliferation test.

Lastly, the present invention provides a method of modulating an autoimmune response, in a patient in need of such treatment, wherein said method comprises administering to said patient a peptide selected from the peptides of the invention, the full-length hsp70 or its active derivatives.

It is to be understood that as a method of modulating it is meant a method for treatment or prevention of the autoimmune disease, which can also be described as a method of vaccination for said disease.

In one embodiment, said peptide should be administered in a medically effective amount, at least once, preferably soon after diagnosis. The peptide may also be administered another two times, preferably at one and six months after the first administration, to provide a booster for the patient.

The treatment is known to be effective when the patient in treatment presents, for example, the capacity to maintain his/her ability to produce the insulin C-peptide. Alternatively, the treatment is known to be effective when the T-cell response of the patient in treatment, in response to hsp70, shows increased interleukin 4, interleukin 10 and interleukin 13 production (Th2 cytokines), for example. The production of other cytokines, like interferon γ and IL2 (Th1 cytokines), can also be evaluated in T-cells of patients following treatment. The modulation of cytokine profile should reflect a shift from a pro-inflammatory T-helper-1 (Th1) response to an anti-inflammatory T-helper-2 (Th2) response, triggered by the hsp70 peptides.

The peptide may be administered to a patient diagnosed with an autoimmune disease, preferably Type 1 Diabetes, Systemic Lupus Erithematosus, Multiple Sclerosis or Rheumatoid Arthritis. More preferably, said autoimmune disease is Type 1 Diabetes. For prevention, the treatment may be given to individuals with a genetic predisposition to one autoimmune disease, for example for individuals who had a family member diagnosed with the same disease.

It is not known why autoimmunity to hsp60, glutamic acid decarboxylase, insulin secretory-granule 38 kDa protein (38 kDa), and other antigens not exclusively expressed in the pancreatic islets should be associated with the autoimmune process leading to Type 1 Diabetes in humans and mice [Honeyman and Harrison (1993) id ibid.; Roep, B. (1996) id ibid.; Rudy, G. et al. (1995) Molecular Medicine, 1:625-633]. Autoimmunity to hsp70 is likely to have a functional role in the Type 1 Diabetes process during childhood. Here the inventors have shown that an immunoreaction to hsp70 can be a useful marker for the detection of Type 1 Diabetes, particularly in children. Potentially the hsp70 protein or its derivatives could be used in a vaccine to treat such children, as well as adults (Table 1D). Such hsp70 vaccine could also be used to treat individuals carrying other autoimmune diseases, like Type 1 Diabetes, Systemic Lupus Erithematosus, Multiple Sclerosis or Rheumatoid Arthritis. More preferably, said autoimmune disease is Type 1 Diabetes.

Intact hsp70 and hsp70 major peptides may be tested as vaccines to prevent the progression of diabetes in NOD mice.

The hsp70 peptides of the present invention can be administered in a variety of ways to modulate the immune response of an individual (e.g., a human, other mammal or other vertebrate). In another embodiment, the protein or peptide is administered as a vaccine which is comprised of at least one hsp70 peptide of the invention, or a portion of it, which is of sufficient size to stimulate the desired immune response.

Alternatively, T-cell vaccination could be used. For that purpose, hsp70 specific, auto-reactive T-cells are isolated from the patient in need of said treatment, and activated in vitro with hsp70 protein or peptides. The responding T-cells are then selected and isolated, expanded, and finally attenuated. The attenuated T-cells are then administered to the patient, as the T-cell vaccine.

Therefore, the method of the present invention can also be used to modify or modulate an individual's response to his or her own cells, in an autoimmune disease. As shown by the inventors, hsp70 protein is involved in Type 1 Diabetes, an autoimmune disease. It is, thus, possible to turn down an individual's immune response, resulting in the individual becoming more tolerant to the protein. It is possible to selectively inhibit or interfere with the ability of immune cells which normally interact with such proteins to do so.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures

Subjects

(a) Type 1 Diabetes patients: 25 children (mean age 10.2±4.2 years), consecutively admitted to the Department of Pediatric Endocrinology of the Hadassah University Hospital (Hebrew University of Jerusalem, Israel), were enrolled in the study, with informed consent obtained from the parents. The mean time elapsed from the time of diagnosis was 3.8 weeks (range 1-8 weeks). The criteria for Type 1 Diabetes diagnosis were: classical clinical symptoms, including a recent history of polyurea, polydipsia, weight loss with or without associated severe ketoacidosis, ketonuria and hyperglycemia (glucose≧200 mg/dl or 11.1 mM/l)

b) Type 2 Diabetes patients: Eleven adult patients (mean age 63±6.6 years) diagnosed as Type 2 Diabetes/NIDDM patients at the Department of Endocrinology, Hadassah University Hospital (Hebrew University of Jerusalem, Israel), provided blood samples with informed consent. In an attempt to control the possible effects of disease severity and insulin therapy, the Type 2 Diabetes patients were all in need of insulin to manage their hyperglycemia. The mean duration of disease of this group was 20.1±7.7 years. Mean time of insulin treatment was 6.6±4.9 years.

c) Healthy Blood Donors: Samples of surplus blood to be used as control were obtained from the Blood Bank (Tel HaShomer Hospital, Tel Aviv, Israel) from 25 healthy adult donors. It was necessary to use most of the control samples from healthy adults because drawing blood from healthy children is unacceptable practice, unless in the below-specified circumstances.

d) Blood sera from healthy children: Blood sera from 10 normoglycemic children (mean age 6.5±4.5 years) were obtained, after parental permission, from children admitted to the Emergency Room of the Hadassah University Hospital (Jerusalem, Israel) for treatment of acute medical conditions.

e) Pediatric T-cell donors: Samples of surplus blood from three children was also obtained, with parental permission, from which T cells were isolated. These children (two females, one male; ages 3, 7 and 10 years old) were not Type 1 diabetes patients, and were admitted to the Department of Pediatrics, Hadassah University Hospital, Hebrew University of Jerusalem (Jerusalem, Israel).

HsD70 Peptides

Hsp70 peptides were prepared in the Biological Services Laboratory of the Weizmann Institute of Science (Rehovot, Israel), using an automated Abimed Synthesizer (Model MAF422, Langenfeld, Germany). The peptides were purified by reverse phase HPLC and their compositions were confirmed by amino acid analysis. The peptides were provided lyophilized and stored at −20° C. Prior to use, the peptides were dissolved in PBS to a concentration of 5-20 μg/ml, and the remaining of the dissolved peptides was stored at −20° C. for further usage. Except for p43, all the hsp70 peptides were 20 amino acids in length, with 5 overlapping amino acids in each side. The amino acid sequences of all of the peptides used herein are shown in Table 2.

T-cell Proliferation Assay

For T-cell proliferation assays, 30-50 ml of peripheral blood was used as described [Abulafia-Lapid et al. (1999) id ibid.]. Anti-coagulation was achieved with [10 IU/ml heparin]. Peripheral blood mononuclear cells (PBMC) were isolated by Ficoll Paque (Pharmacia Biotech, Uppsala Sweden) density centrifugation. The cells were washed with RPMI culture media (Biological Industries, Kibbutz Beit Haemek, Israel), supplemented with 1% Sodium-Pyruvate, 1% L-Glutamine (200 mM), 1% Penicillin/Streptomycin (10,000 U/ml/10,000 mg/ml) and 2% Hepes (1 M, pH 7.3) (all from Biological Industries, Beit Haemek, Israel). PBMC were plated in triplicate or in quadruplicate in 96-well round-bottom micro plates (Falcon, Lincoln Park, N.J., USA) at a cell concentration of 2×10⁵ cells per well in 100 μl RPMI media, with or without the following test antigens: PHA (Murex Diagnostic Ltd. England) 0.3 μg/ml; Tetanus Toxoid (Connaught Lab. Inc, Penn., USA) 5 μg/ml; Candida Albicans 20 μg/ml, recombinant human hsp60, hsp70 and hsp90 (StressGen, Canada), 2-5 μg/ml; and hsp70 peptides, 5-20 μg/ml (synthesized at the Biological Services Laboratory of the Weizmann Institute of Science, Rehovot, Israel, using an automated ABIMED synthesizer AMA422, Langenfeld, Germany). All proliferation assays, including the epitope mapping, were performed in RPMI medium supplemented with 10% autologous serum, and incubated at 37° C. in a 5% CO₂ humidified incubator for 7 days. On day 6, the cells were labeled with 1 μCi/well of ³H-Thymidine. On day 7, the radioactivity was counted using a beta-counter (Packard model 2000).

Proliferation was represented as stimulation index (S.I.), obtained from the ratio between the mean value of proliferation (in cpm) with antigen and the mean value of proliferation without antigen (in cpm). S.I. values were considered positive when greater than or equal to 2.

Determination of Auto-Antibodies

All of the serum samples that were collected were stored in aliquotes at −20° C. and thawed before each assay. Total human IgG anti-hsp60, anti-hsp70, and anti-hsp90, as well as anti-tetanus toxoid proteins were detected by the antibody capture-type enzyme immunoassay ELISA [Maggio, M. T. (1981) Enzyme Immunoassay, CRC Press, Boca Raton, USA]. 96-well micro titer plates (Dynatech) were coated with the protein according to the antibody being tested. Serum samples were distributed at dilutions in the range of 1:50 to 1:70 into the pre-coated wells for the protein detection assay. Thus, any IgG anti-hsp60, anti-hsp70, anti-hsp90, or anti-tetanus toxoid present should be bound by the immobilized peptide or protein. After washing off unbound material, a mouse monoclonal anti-human IgG antibody conjugated to alkaline phosphatase (AP) was added to the wells. After an additional washing step to remove any unbound anti-enzyme reagent, the AP substrate p-nitrophenyl phosphate (pNPP) solution was added. The intensity of the color developed was measured using an Anthos htll ELISA reader at λ=405 nm. The amount of antibody detected was represented as Units of Optical Density at λ=405 nm. Patients and control subjects were assayed in the same experiment.

Statistical Analysis

The InStat 2.01 computer program was used for the statistical analysis. The results obtained from the samples of healthy individuals were used to determine the cutoff for each assay, which was established as the mean plus two standard deviations (SDs). Samples that displayed values above this calculated cutoff were considered positive. The results were presented as the percentage of positive from the total tested group.

In addition, p values were approximated using the two-tailed Fisher's exact test.

### Example 1

T-cell Responses to hsp70

T-cell responses to intact hsp60, hsp70, and hsp90 proteins, and to the recall antigens tetanus toxoid and Candida albicans were compared between the Type 1 Diabetes children (Table 1A), the healthy blood donor subjects (Table 1B) and the Type 2 Diabetes patients (Table 1C).

Table 1: T-cell proliferative response to hsp60, hsp70, or hsp90

TABLE 1A
Type 1 Diabetes children

| | Age | | | | | | |
|---|---|---|---|---|---|---|---|
| Subject | (years) | Weeks* | T.T. | Candida | hsp60 | hsp70 | hsp90 |
| P1 | 16 | 3 | 5.4 | 6.5 | 1.3 | 1.3 | 1.2 |
| P2 | 5 | 5 | 24.0 | 19.2 | 3.6 | 2.0 | 1.0 |
| P3 | 13 | 3 | 34 | 38 | 2.5 | 1.7 | 1 |
| P4 | 9 | 1 | 13.8 | 7.3 | 1.0 | 4.8 | 1.4 |
| P5 | 14 | 3 | 40.6 | 35.2 | 6.0 | 7.3 | 3.8 |
| P6 | 14 | 4 | 6.7 | 3.9 | 3.3 | 5.6 | 1.0 |
| P7 | 1.8 | 3 | 72.8 | 23.5 | 5.5 | 3.3 | 1.1 |
| P8 | 9.5 | 4 | 10.9 | 13.0 | 1.4 | 2.1 | 1.6 |
| P9 | 13 | 3 | 14.2 | 25.4 | 6.1 | 7.8 | 1.0 |
| P10 | 5 | 3 | 39.3 | 4.3 | 2.1 | 14.3 | 1.5 |
| P11 | 9 | 3 | 4.5 | 3.6 | 3.7 | 8.0 | 3.0 |
| P12 | 16 | 3 | 7.1 | 1.7 | 1.9 | 1.0 | 1.0 |
| P13 | 18 | 3 | 25.0 | 15.3 | 13.4 | 8.9 | 1.7 |
| P14 | 10 | 3 | 21.4 | 18.5 | 2.4 | 2.3 | 1.6 |
| P15 | 6 | 2 | 9.2 | 8.1 | 2 | 1 | 2.6 |
| P16 | 7.5 | 3 | 10.1 | 10.5 | 4.6 | 4.9 | 0.8 |
| P17 | 9 | 3 | 4.2 | 6.3 | 2.4 | 1.5 | 1 |
| P18 | 9 | 4 | 10.2 | 6.3 | 3.7 | 3.7 | 1 |
| P19 | 6.5 | 3 | 4.5 | 5.3 | 7.1 | 2 | 1 |
| P20 | 10 | 3 | 17.7 | 13 | 2.7 | 4.4 | 5.1 |
| P21 | 16 | 8 | nd | 23 | 1 | 4.9 | 1.9 |
| P22 | 10.5 | 2 | 5.6 | 7.1 | 3.6 | 4.4 | 3.7 |
| P23 | 7 | 12 | 17.4 | 18 | 3 | 6.2 | n.d. |
| P24 | 9 | 4 | 5.5 | 9.4 | 4.5 | 4.7 | 1.5 |
| P25 | 8 | 8 | 7.7 | 6.9 | 2.1 | 4.4 | 1.5 |
| Mean ± SD | 10.1 ± 3.8 | 3.8 ± 2.3 | 17.2 ± 15.9 | 13.2 ± 9.6 | 3.6 ± 2.6 | 4.5 ± 3.1 | 1.7 ± 1.1 |
| | | | (p = 0.035) | (n.s.) | (p < 0.0001) | (p < 0.0001) | (n.s.) |

*Weeks since diagnosis.
nd = not done.
n.s. = not statistically significant.

TABLE 1B
Healthy subjects

| Sample | Tetanus | | | | |
|---|---|---|---|---|---|
| No. | Toxoid | Candida | hsp60 | hsp70 | hsp90 |
| C1 | 1 | 16 | 1.9 | 2.1 | 2.5 |
| C2 | 50 | 26 | 2.5 | 1.5 | 2.3 |
| C3 | 13.8 | 18.3 | 2.1 | 1 | 1.8 |
| C4 | 2.9 | 2.5 | 1 | 1 | 1 |
| C5 | 8.7 | 6.5 | 2.6 | 1.3 | 4 |
| C6 | 3 | 5 | 1.3 | 1.4 | 1.3 |
| C7 | 15.7 | 8.5 | 1 | 1.1 | 1 |
| C8 | 3.2 | 10.5 | 1.4 | 1.5 | 2.2 |
| C9 | 10.6 | 12.3 | 2 | 1 | 1.8 |
| C10 | 4.3 | 7.1 | 1.75 | 1 | 1.4 |
| C11 | 11.3 | 17.8 | 2.9 | 1 | 1 |
| C12 | 3.5 | 3.6 | 2.9 | 1.5 | 1 |
| C13 | 5.5 | 11.1 | 2.3 | 1.7 | 1.9 |
| C14 | 9 | 4.5 | 1 | 1 | 1 |
| C15 | 19.3 | 13.9 | 1.3 | 1.6 | 1 |
| C16 | 3 | 6 | 1 | 1 | 1.5 |
| C17 | 6.6 | 8.8 | 2.5 | 1.5 | 1.2 |
| C18 | 4.3 | 8.9 | 3.5 | 3.2 | 2.4 |
| C19 | 2.5 | 5.1 | 1.3 | 1.3 | 2.4 |
| C20 | 2.4 | 4.6 | 1 | 1.5 | 2 |
| C21 | 3.4 | 7.3 | 1.9 | 1 | 1 |
| C22 | 10.7 | 25 | 1 | 3.1 | 1.1 |
| C23 | 2.9 | 2.5 | 1 | 1 | 1 |
| C24 | 2.7 | 3.7 | 1.5 | 1.9 | 1 |
| C25 | 11.1 | 77 | 11 | 22 | 11 |
| C26* | 7.3 | 16 | 1.25 | 1.1 | 1 |
| C27* | 3.5 | 3.6 | 1 | 1 | 1 |
| C28* | 2.2 | 2.1 | 1 | 1 | 1 |
| Mean ± SD | 8.01 ± 9.3 | 9.4 ± 6.4 | 1.7 ± 0.72 | 1.44 ± 0.6 | 1.52 ± 0.7 |

C26*-C28*: Pediatric T-cell donors

TABLE 1C
Type 2 Diabetes

| Sample | Age | Tetanus | Candi- | | | |
|---|---|---|---|---|---|---|
| No. | (years) | Toxoid | da | hsp60 | hsp70 | hsp90 |
| N1 | 75 | 2.7 | 5.1 | 1.1 | 1.3 | 1.2 |
| N2 | 63 | 10.1 | nd | 1.3 | 1 | 1 |
| N3 | 65 | 8.1 | nd | 2.6 | 1 | 1 |
| N4 | 60 | Nd | 45 | 1.5 | 1.2 | 1.5 |
| N5 | 63 | 1 | 1 | 1 | 1.1 | 1.2 |
| N6 | 60 | Nd | 5.2 | 2.4 | 2.1 | 1.8 |
| N7 | 72 | 3.7 | 2.5 | 1 | 1 | 1 |
| N8 | 60 | 3.6 | 5.2 | 1.2 | 1.1 | 1.3 |
| N9 | 50 | 2.5 | nd | 3.3 | 3.9 | 1 |
| N10 | 66 | 3 | nd | 1.7 | 4.6 | 5.7 |
| N11 | 60 | 4.6 | 2.5 | 1.8 | 1.45 | 1 |
| Mean ± | 63 ± | 4.4 ± | 9.5 ± | 1.7 ± 0.75 | 1.8 ± 1.26 | 1.6 ± 1.38 |
| SD | 6.6 | 2.9 | 15.7 | | | |

nd = not done.

TABLE 1D
T-cell responses of Type 1 Diabetes adult patients

| | | | Time since | | | | | |
|---|---|---|---|---|---|---|---|---|
| | | | diagnosis | Tetanus | | | | |
| Patient | Age | Gender | (weeks) | toxoid | Candida | hsp60 | hsp70 | hsp90 |
| AP1 | 25 | M | 12 | 1 | — | 1 | 1.70 | 1.13 |
| AP2 | 20.1 | F | 19 | 7.8 | nd | 1.2 | 2.09 | 1.22 |
| AP3 | 20 | M | 10 | 8.4 | — | 2.1 | 3.20 | 1.39 |
| AP4 | 18 | M | 8 | 3.8 | nd | 1.2 | 2.8 | 1.20 |
| AP5 | 19 | F | 16 | 5.9 | nd | 1 | 2.00 | — |
| AP6 | 25 | M | 12 | 3.2 | 1.5 | 1.1 | 2.1 | 1.40 |
| AP7 | 22 | M | 12 | 70 | 8.3 | 1 | 4.3 | 2.20 |
| Mean ± SD | 21.3 ± 2.59 | | 12.71 ± 3.41 | 14.3 ± 22.86 | 4.9 ± 3.4 | 1.22 ± 0.36 | 2.6 ± 0.84 | 1.4 ± 0.3 |

Type 1 Diabetes patients were identified as P1 to P25, healthy subjects (control) as C1-C28, and Type 2 Diabetes patients as N1-N11. Three pediatric controls are C26-C28. Adult patients are AP1-AP1 (Table 1D).

T-cells were isolated from 25 children newly diagnosed with Type 1 Diabetes (Table 1A), 28 healthy subjects (Table 1B), and 11 Type 2 Diabetes patients (Table 1C). The subjects were tested for their proliferative responses to hsp60, hsp70, and hsp90 and also to recall antigen Tetanus Toxoid and Candida albicans. The responses, shown as Stimulation Index (S.I.), represent the ratio of the mean T-cell response with antigen to the T-cell response without antigen. A value of S.I. equal or greater than 2 was considered positive. T-cell response was evidenced by cell proliferation measured in cpm. Statistical analysis was performed using the InState 2.01 computer program, and p-values were approximated by the Krusal-Wallis nonparametric ANOVA test.

Surprisingly, T-cell responses of Type 1 Diabetes children to hsp70 (Table 1A) were significantly higher than those of the other two groups. There were significantly more responding Type 1 Diabetes children (20 of 25; 85%) than healthy blood donors (4 out of 28; 14%) (p=0.0006) or Type 2 Diabetes patients (3 out of 11; 27%) (p=0.0006). The degree of responsiveness to hsp70 was also higher in the Type 1 Diabetes group (mean S.I.=4.5±3.1) when compared to that of the Type 2 Diabetes group (mean S.I.=1.8±1.3) or to the healthy blood donors (mean S.I.=1.4±0.6; p<0.0001). Thus, recently diagnosed Type 1 Diabetes children had an enhanced T-cell proliferative response to hsp70. Although 20 of the 25 Type 1 Diabetes children responded to hsp60, this response was lower (mean of S.I.=3.6±2.6) than the response to hsp70 (mean of S.I.=4.5±3.1). Interestingly, three Type 1 Diabetes patients (P4, P8 and P21) responded exclusively to hsp70 and not to hsp60 (or hsp90). It is important to highlight that the difference between the responses of Type 1 Diabetes patients and healthy individuals to hsp70 (SI_{IDDM}=40±3.1 versus SI_{healthy}=1.45±0.6, or 85% versus 13%, respectively) is higher than the difference of their responses to hsp60 (SI_{IDDM}=3.6±2.6 versus SI_{healthy}=1.79±0.74, or 85% versus 39%, respectively). In sum, these results demonstrate that the T-cell response to hsp70 is a more distinctive parameter for the diagnosis and treatment of Type 1 Diabetes.

In contrast, 5 out of 24 (21%) Type 1 Diabetes children tested responded to hsp90 (mean S.I.=1.7±1.2). In the other two groups, there were 7 out of 28 healthy subjects (25%) (mean S.I.=1.52±0.7) and 1 out of 11 (9%) Type 2 Diabetes subjects (mean S.I.=1.6±1.4) that responded to hsp90.

The three groups tested, shown in Tables 1A, 1B and 1C responded similarly to the Candida antigen. The responses of the Type 1 Diabetes children (mean S.I.=17.2±16.1, Table 1A) to the tetanus toxoid were higher than those of the healthy subjects (S.I.=9.4±6.4, p=0.035; Table 1B), whereas the responses of the Type 2 Diabetes subjects were lower than the latter (S.I.=4.4±2.9, Table 1C). This result was expected, since the response to tetanus toxoid is usually higher in younger patients due to the fact that these individuals likely received a booster shot immunization more recently than older individuals. Therefore, the result of the Type 2 Diabetes group was the lowest, probably because they were older (mean age of 63±6.6) than the subjects in the other two groups, and thus even less likely to have received a tetanus toxoid booster shot in the recent past.

It is important to mention that, although the sample size is small, the results obtained for T cell responses from C26-C28 (healthy children) to hsp60, hsp70 and hsp90 were comparable to that of C1-C25 (healthy adults).

From the results shown in Table 1D it can be seen that these adult Type 1 patients, whose age range was 18-23, nicely responded to hsp70 within 12 weeks from diagnosis (about 86%, mean=2.6+0.84). A response to hsp60 treatment is not seen, possibly because these patients were tested after more than 12 weeks. This means that the response to hsp70 is longer. No response to hsp90 was observed, possibly because the test was late, as compared to children. It appears from these preliminary results that anti-hsp70 antibodies may be better than anti-hsp60 and anti-hsp90.

Dynamics of the hsp70 and hsp60 Responses

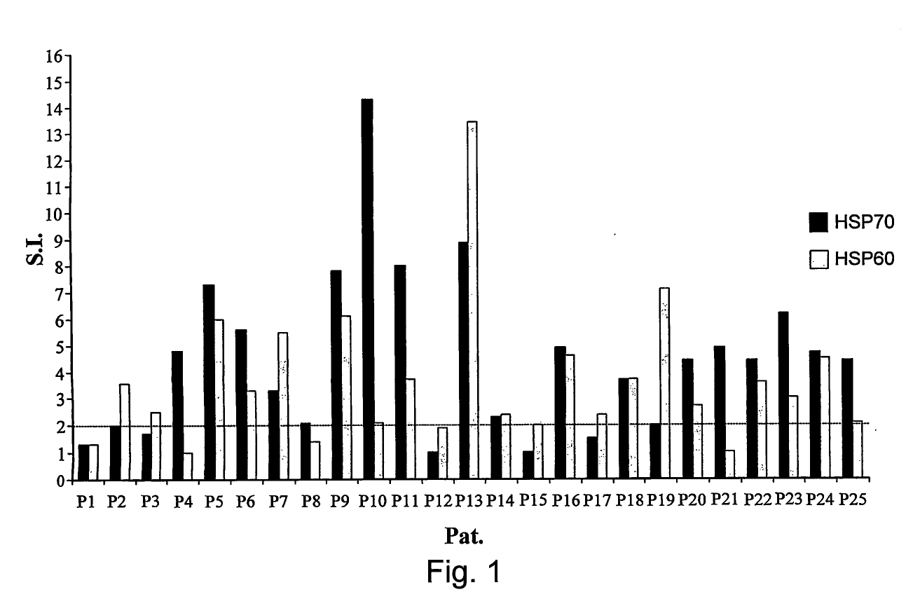

In FIG. 1, the responsiveness of the Type 1 Diabetes children to hsp70 can be compared with that to hsp60, and the magnitude of the T-cell responses can be appreciated (FIG. 1 and Table 1A). Amongst the 20 Type 1 Diabetes subjects that responded to hsp70, 17 (85%) also responded to hsp60, and from these, 12 had their hsp70 response either higher or equal to the hsp60 response (FIG. 1). It is important to note that the mean S.I. value of the hsp70 response was higher than that of the hsp60 response, and from those patients that responded to both hsp70 and hsp60, the majority responded better to hsp70. This strengthens the inventor's finding that hsp70 is an ideal treatment and diagnostic tool for Type 1 Diabetes.

### Example 2

Epitope Mapping of the hsp70 Peptides

In order to determine the spectrum of the hsp70 peptides that were recognized in the Type 1 Diabetes children, T-cell proliferative responses to the 43 overlapping hsp70 peptides (Table 2) were assayed (FIG. 2).

TABLE 2

Overlapping peptides of the human hsp70 molecule

Peptide

| | | | |
|---|---|---|---|
| Sequence ID | Number | Position | Sequence |

| | | | |
|---|---|---|---|
| SEQ. ID. NO. 1 | p1 | 1-20 | MAKAAAVGIDLGTTYSCVGV |
| SEQ. ID. NO. 2 | p2 | 16-35 | SCVGVFQHGKVEIIANDQGN |
| SEQ. ID. NO. 3 | p3 | 31-50 | NDQGNRTTPSYVAFTDTERL |
| SEQ. ID. NO. 4 | p4 | 46-65 | DTERLIGDAAKNQVALNPQN |
| SEQ. ID. NO. 5 | p5 | 61-80 | LNPQNTVFDAKRLIGRKFGD |
| SEQ. ID. NO. 6 | p6 | 76-95 | RKFGDPVVQSDMKHWPFQVI |
| SEQ. ID. NO. 7 | p7 | 91-110 | PFQVINDGDKPKVQVSYKGE |
| SEQ. ID. NO. 8 | p8 | 106-125 | SYKGETKAFYPEEISSMVLT |
| SEQ. ID. NO. 9 | p9 | 121-140 | SMVLTKMKEIAEAYLGYPVT |
| SEQ. ID. NO. 10 | p10 | 136-155 | GYPVTNAVITVPAYFNDSQR |
| SEQ. ID. NO. 11 | p11 | 151-170 | NDSQRQATKDAGVIAGLNVL |
| SEQ. ID. NO. 12 | p12 | 166-185 | GLNVLRIINEPTAAAIAYGL |
| SEQ. ID. NO. 13 | p13 | 181-199 | IAYGLDRTGKGERNVLIFDL |
| SEQ. ID. NO. 14 | p14 | 195-214 | LIFDLGGGTFDVSILTIDDG |
| SEQ. ID. NO. 15 | p15 | 210-229 | TIDDGIFEVKATAGDTHLGG |
| SEQ. ID. NO. 16 | p16 | 225-244 | THLGGEDFDNRLVNHFVEEF |
| SEQ. ID. NO. 17 | p17 | 240-259 | FVEEFKRKHKKDISQNKRAV |
| SEQ. ID. NO. 18 | p18 | 255-275 | NKRAVRRLRTACERAKRTLS |
| SEQ. ID. NO. 19 | p19 | 271-290 | KRTLSSSTQASLEIDSLFEG |
| SEQ. ID. NO. 20 | p20 | 286-305 | SLFEGIDFYTSITRARFEEL |
| SEQ. ID. NO. 21 | p21 | 301-320 | RFEELCSDLFRSTLEPVEKA |
| SEQ. ID. NO. 22 | p22 | 316-335 | PVEKALRDAKLDKAQIHDLV |
| SEQ. ID. NO. 23 | p23 | 331-350 | IHDLVLVGGSTRIPKVQKLL |
| SEQ. ID. NO. 24 | p24 | 346-365 | VQKLLQDFFNGRDLNKSINP |
| SEQ. ID. NO. 25 | p25 | 361-380 | KSINPDEAVGYGAAVQAAIL |
| SEQ. ID. NO. 26 | p26 | 376-395 | QAAILMGDKSENVQDLLLLD |
| SEQ. ID. NO. 27 | p27 | 391-410 | LLLLDVAPLSLGLETAGGVM |
| SEQ. ID. NO. 28 | p28 | 406-425 | AGGVMTALIKRNSTIPTKQT |
| SEQ. ID. NO. 29 | p29 | 421-440 | PTKQTQIFTTYSDNQPGVLI |
| SEQ. ID. NO. 30 | p30 | 436-455 | PGVLIQVYEGERAMTKDNNL |
| SEQ. ID. NO. 31 | p31 | 451-470 | KDNNLLGRFELSGIPPAPGV |
| SEQ. ID. NO. 32 | P32 | 466-485 | PAPGVPQIEVTFDIDANGIL |
| SEQ. ID. NO. 33 | p33 | 481-500 | ANGILNVTATDKSTGKANKI |
| SEQ. ID. NO. 34 | p34 | 496-515 | KANKITITNDKGRLSKEEIE |
| SEQ. ID. NO. 35 | p35 | 511-530 | KEEIERMVQEAEKYKAEDEV |
| SEQ. ID. NO. 36 | p36 | 526-545 | AEDEVQRERVSAKNALESYA |
| SEQ. ID. NO. 37 | p37 | 541-560 | LESYAFNMKSAVEDEGLKGK |
| SEQ. ID. NO. 38 | p38 | 556-575 | GLKGKISEADKKKVLDKCQE |
| SEQ. ID. NO. 39 | p39 | 571-590 | DKCQEVISWLDANTLAEKDE |
| SEQ. ID. NO. 40 | p40 | 586-605 | AEKDEFEHKRKELEQVCNPI |
| SEQ. ID. NO. 41 | p41 | 601-620 | VCNPIISGLYQGAGGPGPGG |
| SEQ. ID. NO. 42 | p42 | 616-635 | PGPGGFGAQGPKGGSGSGPT |
| SEQ. ID. NO. 43 | p43 | 631-640 | GSGPTIEEVD |

In FIG. 2, seven Type 1 Diabetes patients (P3, P16, P18, P20, P21, P23 and P24 from Table 1A) were tested for their responsiveness to the 43 hsp70 peptides (Table 2). Reactivity was measured as T-cell response to each of the peptides in a proliferation assay as described above. Peptides were considered immunogenic when at least 3 out of the 7 patients tested had a positive response. As before, a S.I. value of 2 and greater was considered positive. Amongst the seven subject samples tested, there was reactivity to nine of the 43 peptides. More precisely, the nine peptides to which there was reactivity were: p1, p12, p15, p16, p19, p27, p29, p34 and p35 (FIG. 2). Patients P24 and P23 reacted to six and seven out of these nine peptides, respectively. Because six out of seven of the Type 1 Diabetes subjects tested responded to peptide p27 (residues 391-410, SEQ. ID. NO.27), five responded to peptide p35 (residues 511-530, SEQ. ID. NO.35), and five (P21, P23 and P24, see FIG. 2A, and two Type 1 Diabetes adult patients, data not shown) responded to p1 (residues 1-20, SEQ. ID. NO.1), these three peptides were considered the major antigenic peptides in the hsp70 protein. In conclusion, multiple hsp70 peptides appear to be recognized by the Type 1 Diabetes population, amongst which, three of them (p1, p27 and p35) seem to be its hallmark and harbor major antigenic sites.

### Example 3

Auto-Antibodies to hsp60, hsp70, and hsp90

Levels of IgG antibodies to hsp60, hsp70, and hsp90 were measured in the sera of 20 Type 1 Diabetes children (P1-P5, P7-P21; Table 3A) and two control groups, 10 normoglycemic children (Table 3B) and 15 healthy adult blood donors (Table 3C). The level of the antibodies was scored as positive when it was greater than the cut-off level (cut-off levels were established based on the mean value of antibody levels from the normoglycemic children plus two standard deviations).

TABLE 3A
IgG antibodies to hsp60, hsp70 and hsp90 in Type 1 diabetes
children

| | | Diabetes | | | |
|---|---|---|---|---|---|
| | | duration | | | |
| Subject | Age/Sex | (Weeks*) | Hsp60 | Hsp70 | Hsp90 |
| P1 | 16/F | 3 | 1.04 | 0.53 | 0.66 |
| P2 | 5/F | 5 | 0.86 | 2 | 0.68 |
| P3 | 13/F | 3 | 0.45 | 0.28 | 0.45 |
| P4 | 9/F | 1 | 0.67 | 0.37 | 0.45 |
| P5 | 14/M | 3 | 0.77 | 0.7 | 0.44 |
| P7 | 1.8/M | 3 | 0.29 | 0.26 | 0.35 |
| P8 | 9.5/M | 4 | 0.69 | 0.34 | 0.59 |
| P9 | 13/M | 3 | 0.57 | 0.3 | 0.37 |
| P10 | 5/F | 3 | 0.46 | 0.26 | 0.45 |
| P11 | 9/F | 3 | 0.34 | 0.25 | 0.39 |
| P12 | 16/M | 3 | 0.46 | 0.27 | 0.39 |
| P13 | 18/M | 3 | 1.25 | 0.75 | 0.82 |
| P14 | 10/M | 3 | 0.51 | 0.29 | 0.45 |
| P15 | 6/F | 2 | 0.72 | 0.33 | 0.27 |
| P16 | 7.5/F | 3 | 0.76 | 1.22 | 0.6 |
| P17 | 9/F | 3 | 0.54 | 0.28 | 0.43 |
| P18 | 9/F | 4 | 0.49 | 0.29 | 0.37 |
| P19 | 6.5/F | 3 | 0.35 | 0.23 | 0.38 |
| P20 | 10/M | 3 | 0.39 | 0.88 | 0.33 |
| P21 | 16/F | 8 | 0.73 | 0.44 | 0.56 |
| Mean ± SD | 10.1 ± 4.1 | 3.3 ± 1.2 | 0.6 ± | 0.5 ± 0.41 | 0.47 ± 0.13 |
| | | | 0.23 | | |
| Cut-off | | | 0.67 | 0.53 | 0.64 |
| Positive | | | 45% | 30% | 15% |

*weeks since diagnosis

TABLE 3B
IgG antibodies to hsp60, hsp70 and hsp90 in pediatric
control serum donors

| Subject | Age/Sex | Hsp60 | Hsp70 | Hsp90 |
|---|---|---|---|---|
| C1 | 7/M | 0.61 | 0.41 | 0.41 |
| C2 | 3/M | 0.39 | 0.34 | 0.54 |
| C3 | 12/M | 0.35 | 0.45 | 0.47 |
| C4 | 13/M | 0.28 | 0.26 | 0.41 |
| C5 | 10/M | 0.47 | 0.47 | 0.56 |
| C6 | 0.5/M | 0.39 | 0.35 | 0.3 |
| C7 | 3/M | 0.43 | 0.27 | 0.3 |
| C8 | 2/M | 0.5 | 0.37 | 0.55 |
| C9 | 4/F | 0.4 | 0.17 | 0.28 |
| C10 | 10/M | 0.67 | 0.44 | 0.5 |
| Mean ± SD | 6.45 ± 4.3 | 0.45 ± 0.11 | 0.35 ± 0.09 | 0.43 ± 0.1 |
| Cut-off | | 0.67 | 0.53 | 0.64 |
| Positive | | 10% | 0 | 0 |

TABLE 3C
IgG antibodies to hsp60, hsp70 and hsp90 in healthy
adult blood donors

| Subject | Hsp60 | Hsp70 | Hsp90 |
|---|---|---|---|
| AC1 | 0.53 | 0.30 | 0.42 |
| AC2 | 0.57 | 0.35 | 0.45 |
| AC3 | 0.76 | 0.70 | 0.41 |
| AC4 | 0.75 | 0.50 | 0.74 |
| AC5 | 0.57 | 0.35 | 0.47 |
| AC6 | 0.38 | 0.26 | 0.31 |
| AC7 | 0.52 | 0.44 | 0.49 |
| AC8 | 0.62 | 0.41 | 0.45 |
| AC9 | 0.63 | 0.46 | 0.49 |
| AC10 | 0.89 | 0.52 | 0.61 |
| AC11 | 0.58 | 0.38 | 0.44 |
| AC12 | 0.52 | 0.35 | 0.3 |
| AC13 | 0.59 | 0.5 | 0.47 |
| AC14 | 0.64 | 0.52 | 0.40 |
| AC15 | 0.67 | 0.41 | 0.49 |
| Mean ± SD | 0.61 ± 0.12 | 0.43 ± 0.1 | 0.46 ± 0.1 |
| Cut-off | 0.85 | 0.64 | 0.67 |
| Positive | 6.6% | 6.6% | 6.6% |

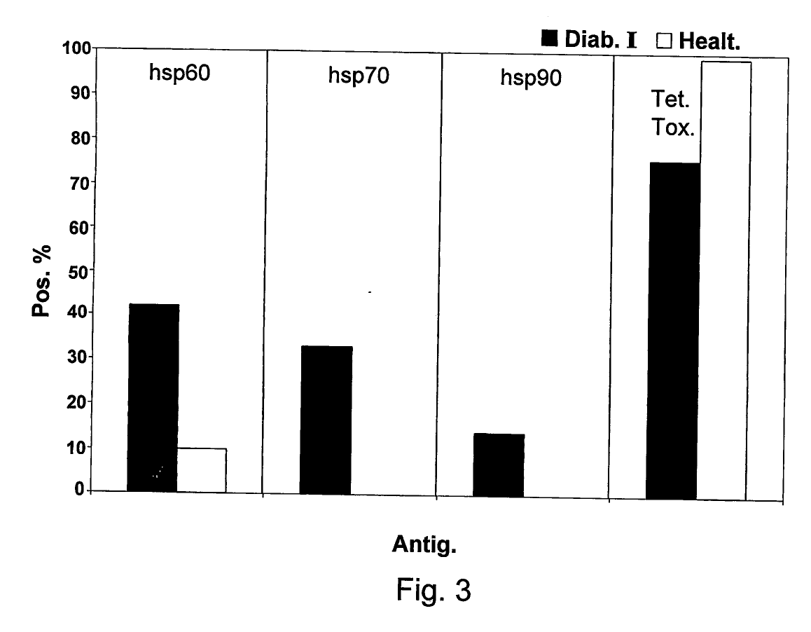

Of the Type 1 Diabetes children, 45% (9 out of 20) were positive to hsp60, 30% (6 out of 20) were positive to hsp70, and 15% (3 out of 20) were positive to hsp90 (FIG. 3). Out of the nine Type 1 Diabetes children positive to hsp60, five were also positive to hsp70. In contrast, of the 10 healthy children, only one (10%) was positive to hsp60, and none were positive to hsp70 or to hsp90 (FIG. 3). Among the healthy adult blood donors, only 6% were positive to hsp60, hsp70 and hsp90. It is remarkable that although only 30% of the Type 1 Diabetes children were positive to hsp70, none of the healthy children were sero-positive to hsp70. This result shows that being sero-positive to hsp70 is one good indicator of Type 1 Diabetes in children. Antibodies to tetanus toxoid were measured as control, and were comparably high in both Type 1 Diabetes and control groups.

There was no correlation between the IgG antibody levels and the magnitude of the T-cell response to these proteins.

### Example 4

Hsp70 Vaccination

Groups of 10 female NOD mice are treated at age 4-6 weeks with 100 μg of hsp70 peptides p1, p27 and p35, and IFA (incomplete freund's adjuvant) as a control group. The peptides are emulsified in oil (IFA). The NOD mice are injected subcutaneously. In order to examine development of hyperglycemia, blood glucose level may be monitored every two weeks using glucose analyzer.

1. A peptide selected from the group consisting of the peptides denoted by SEQ. ID. NO.1, SEQ. ID. NO.2, SEQ. ID. NO.3, SEQ. ID. NO.4, SEQ. ID. NO.5, SEQ. ID. NO.6, SEQ. ID. NO.7, SEQ. ID. NO.8, SEQ. ID. NO.9, SEQ. ID. NO.10, SEQ. ID. NO.11, SEQ. ID. NO.12, SEQ. ID. NO.13, SEQ. ID. NO.14, SEQ. ID. NO.15, SEQ. ID. NO.16, SEQ. ID. NO.17, SEQ. ID. NO.18, SEQ. ID. NO.19, SEQ. ID. NO.20, SEQ. ID. NO.21, SEQ. ID. NO.22, SEQ. ID. NO.23, SEQ. ID. NO.24, SEQ. ID. NO.25, SEQ. ID. NO.26, SEQ. ID. NO.27, SEQ. ID. NO.28, SEQ. ID. NO.29, SEQ. ID. NO.30, SEQ. ID. NO.31, SEQ. ID. NO.32, SEQ. ID. NO.33, SEQ. ID. NO.34, SEQ. ID. NO.35, SEQ. ID. NO.36, SEQ. ID. NO.37, SEQ. ID. NO.38, SEQ. ID. NO.39, SEQ. ID. NO.40, SEQ. ID. NO.41, SEQ. ID. NO.42 and SEQ. ID. NO.43, and salts, analogues and functional derivatives thereof.

2. A peptide according to claim 1, wherein said functional derivatives consist of chemical modifications to amino acid side chains and/or the carboxyl and/or amino moieties of said peptides.

3. A peptide according to claim 1, selected from the group consisting of the peptides SEQ. ID. NO.1, SEQ. ID. NO.12, SEQ. ID. NO.15, SEQ. ID. NO.16, SEQ. ID. NO.19, SEQ. ID. NO.27, SEQ. ID. NO.29, SEQ. ID. NO.34 and SEQ. ID. NO.35.

4. A peptide according to claim 3, selected from the group consisting of peptides SEQ. ID. NO.1, SEQ. ID. NO.27 and SEQ. ID. NO.35.

5. A pharmaceutical composition comprising at least one peptide according to claim 1 and optionally comprising a pharmaceutically acceptable carrier.

6. A pharmaceutical composition comprising at least one peptide according to claim 3 and optionally comprising a pharmaceutically acceptable carrier.

7. A pharmaceutical composition comprising at least one peptide according to claim 4 and optionally comprising a pharmaceutically acceptable carrier.

8. The pharmaceutical composition according to claim 6 for the prevention or treatment of an autoimmune disease.

9. A pharmaceutical composition according to claim 8, wherein said autoimmune disease is Type 1 Diabetes, Systemic Lupus Erithematosus, Multiple Sclerosis or Rheumatoid Arthritis.

10. A pharmaceutical composition according to claim 9, wherein said autoimmune disease is Type 1 Diabetes.

11. A method for diagnosing the occurrence or incipience of an autoimmune disease in a patient by use of a peptide as defined in claim 1.

12. The method according to claim 11, wherein said autoimmune disease is Type 1 Diabetes, Systemic Lupus Erithematosus, Multiple Sclerosis or Rheumatoid Arthritis.

13. The method according to claim 12, wherein said autoimmune disease is Type 1 Diabetes.

14. The method according to claim 11, wherein said method comprises testing a blood or urine sample of said patient for the presence of antibodies or T-cells which are immunologically reactive to human hsp70 by contacting said sample with at least one peptide as defined in claim 3 or 4, and detecting an immunoreaction between said sample and said peptide, wherein the presence of such immunoreaction indicates the presence of anti-hsp70 antibodies or of a T-cell, indicating an increased probability of the presence or incipience of an autoimmune disease.

15. The method according to claim 14, wherein said patient is tested for the presence of anti-hsp70 antibodies.

16. The method according to claim 14, wherein said immunoreaction is detected by radioimmunoassay.

17. The method according to claim 14, wherein said immunoreaction is detected by an ELISA test.

18. The method according to claim 14, wherein said patient is tested for the presence of a T-cell which immunoreacts with hsp70.

19. The method, wherein said patient is tested for the presence of a T-cell which immunoreacts with hsp70, and wherein said method comprises the steps of:
(a) preparing a mononuclear cell fraction containing T-cells from a blood sample obtained from said patient;
(b) adding to said mononuclear cell fraction at least one antigen selected from the peptides defined in claim 3;
(c) incubating said cell fraction in the presence of said antigen for a suitable period of time and under suitable culture conditions;
(d) adding a labeled nucleotide to the incubated cell culture of (c) at a suitable time before the end of said incubation period to provide for the incorporation of said labeled nucleotide into the DNA of proliferating T-cells; and
(e) determining by suitable means the amount of proliferating T-cells by analysis of the amount of labeled nucleotide incorporated into said T-cells.

20. A kit for the diagnosis of an autoimmune disease by testing for the presence of anti-hsp70 antibodies, wherein said kit comprises the following components:
(f) At least one antigen selected from peptides as defined in claim 3; and
(g) a tagged antibody capable of recognizing the non-variable region of said anti-hsp70 antibodies.

21. The kit according to claim 20, wherein said autoimmune disease is Type 1 Diabetes, Systemic Lupus Erithematosus, Multiple Sclerosis or Rheumatoid Arthritis.

22. The kit according to claim 21, wherein said autoimmune disease is Type 1 Diabetes.

23. A kit for the diagnosis of an autoimmune disease by testing for the presence of a T-cell which immunoreacts with hsp70, wherein said kit comprises the following components:
(h) at least one antigen selected from the peptides as defined in claim 3;
(i) a suitable medium for culture of lymphocytes (T-cells); and
(j) a labeled nucleotide for a T-cell proliferation test.

24. The kit according to claim 23, wherein said autoimmune disease is Type 1 Diabetes, Systemic Lupus Erithematosus, Multiple Sclerosis or Rheumatoid Arthritis.

25. The kit according to claim 24, wherein said autoimmune disease is Type 1 Diabetes.

26. A method of modulating an immune response, in a patient in need of such treatment, wherein said method comprises administering to said patient a peptide selected from the peptides as defined in claim 3.

27. The method according to claim 26, wherein said method comprises administering the peptide in a medically effective amount, at least once, to the patient in need of such treatment.


SEQUENCE LISTING (43 sequences)

SEQ ID NO: 1 - protein, 20 aa, Homo sapiens
MAKAAAVGIDLGTTYSCVGV

SEQ ID NO: 2 - protein, 20 aa, Homo sapiens
SCVGVFQHGKVEIIANDQGN

SEQ ID NO: 3 - protein, 20 aa, Homo sapiens
NDQGNRTTPSYVAFTDTERL

SEQ ID NO: 4 - protein, 20 aa, Homo sapiens
DTERLIGDAAKNQVALNPQN

SEQ ID NO: 5 - protein, 20 aa, Homo sapiens
LNPQNTVFDAKRLIGRKFGD

SEQ ID NO: 6 - protein, 20 aa, Homo sapiens
RKFGDPVVQSDMKHWPFQVI

SEQ ID NO: 7 - protein, 20 aa, Homo sapiens
PFQVINDGDKPKVQVSYKGE

SEQ ID NO: 8 - protein, 20 aa, Homo sapiens
SYKGETKAFYPEEISSMVLT

SEQ ID NO: 9 - protein, 20 aa, Homo sapiens
SMVLTKMKEIAEAYLGYPVT

SEQ ID NO: 10 - protein, 20 aa, Homo sapiens
GYPVTNAVITVPAYFNDSQR

SEQ ID NO: 11 - protein, 20 aa, Homo sapiens
NDSQRQATKDAGVIAGLNVL

SEQ ID NO: 12 - protein, 20 aa, Homo sapiens
GLNVLRIINEPTAAAIAYGL

SEQ ID NO: 13 - protein, 20 aa, Homo sapiens
IAYGLDRTGKGERNVLIFDL

SEQ ID NO: 14 - protein, 20 aa, Homo sapiens
LIFDLGGGTFDVSILTIDDG

SEQ ID NO: 15 - protein, 20 aa, Homo sapiens
TIDDGIFEVKATAGDTHLGG

SEQ ID NO: 16 - protein, 20 aa, Homo sapiens
THLGGEDFDNRLVNHFVEEF

SEQ ID NO: 17 - protein, 20 aa, Homo sapiens
FVEEFKRKHKKDISQNKRAV

SEQ ID NO: 18 - protein, 20 aa, Homo sapiens
NKRAVRRLRTACERAKRTLS

SEQ ID NO: 19 - protein, 20 aa, Homo sapiens
KRTLSSSTQASLEIDSLFEG

SEQ ID NO: 20 - protein, 20 aa, Homo sapiens
SLFEGIDFYTSITRARFEEL

SEQ ID NO: 21 - protein, 20 aa, Homo sapiens
RFEELCSDLFRSTLEPVEKA

SEQ ID NO: 22 - protein, 20 aa, Homo sapiens
PVEKALRDAKLDKAQIHDLV

SEQ ID NO: 23 - protein, 20 aa, Homo sapiens
IHDLVLVGGSTRIPKVQKLL

SEQ ID NO: 24 - protein, 20 aa, Homo sapiens
VQKLLQDFFNGRDLNKSINP

SEQ ID NO: 25 - protein, 20 aa, Homo sapiens
KSINPDEAVGYGAAVQAAIL

SEQ ID NO: 26 - protein, 20 aa, Homo sapiens
QAAILMGDKSENVQDLLLLD

SEQ ID NO: 27 - protein, 20 aa, Homo sapiens
LLLLDVAPLSLGLETAGGVM

SEQ ID NO: 28 - protein, 20 aa, Homo sapiens
AGGVMTALIKRNSTIPTKQT

SEQ ID NO: 29 - protein, 20 aa, Homo sapiens
PTKQTQIFTTYSDNQPGVLI

SEQ ID NO: 30 - protein, 20 aa, Homo sapiens
PGVLIQVYEGERAMTKDNNL

SEQ ID NO: 31 - protein, 20 aa, Homo sapiens
KDNNLLGRFELSGIPPAPGV

SEQ ID NO: 32 - protein, 20 aa, Homo sapiens
PAPGVPQIEVTFDIDANGIL

SEQ ID NO: 33 - protein, 20 aa, Homo sapiens
ANGILNVTATDKSTGKANKI

SEQ ID NO: 34 - protein, 20 aa, Homo sapiens
KANKITITNDKGRLSKEEIE

SEQ ID NO: 35 - protein, 20 aa, Homo sapiens
KEEIERMVQEAEKYKAEDEV

SEQ ID NO: 36 - protein, 20 aa, Homo sapiens
AEDEVQRERVSAKNALESYA

SEQ ID NO: 37 - protein, 20 aa, Homo sapiens
LESYAFNMKSAVEDEGLKGK

SEQ ID NO: 38 - protein, 20 aa, Homo sapiens
GLKGKISEADKKKVLDKCQE

SEQ ID NO: 39 - protein, 20 aa, Homo sapiens
DKCQEVISWLDANTLAEKDE

SEQ ID NO: 40 - protein, 20 aa, Homo sapiens
AEKDEFEHKRKELEQVCNPI

SEQ ID NO: 41 - protein, 20 aa, Homo sapiens
VCNPIISGLYQGAGGPGPGG

SEQ ID NO: 42 - protein, 20 aa, Homo sapiens
PGPGGFGAQGPKGGSGSGPT

SEQ ID NO: 43 - protein, 10 aa, Homo sapiens
GSGPTIEEVD